(19) United States
(12) Patent Application Publication
(10) Pub. No.: US 2015/0133778 A1
(43) Pub. Date: May 14, 2015

(54) Diagnostic Device for Dermatology with Merged OCT and Epiluminescence Dermoscopy

(71) Applicant: Medlumics S.L., Tres Cantos-Madrid (ES)

(72) Inventors: Alejandro Barriga Rivera, Sevilla (ES); José Luis Rubio Guivernau, Madrid (ES); Eduardo Margallo Balbás, Madrid (ES)

(73) Assignee: Medlumics S.L., Tres Cantos-Madrid (ES)

(21) Appl. No.: 14/530,054

(22) Filed: Oct. 31, 2014

Related U.S. Application Data
(60) Provisional application No. 61/899,673, filed on Nov. 4, 2013.

(51) Int. Cl. A61B 5/00; G01B 9/02

(52) U.S. Cl. CPC A61B 5/444; G01B 9/02091; A61B 5/0066; A61B 5/0077; A61B 5/7246; A61B 5/7425; A61B 5/0013; A61B 5/0017; A61B 5/0022; A61B 2576/02
USPC 600/427

(57) ABSTRACT

Systems and methods for use of the imaging system are presented. In an embodiment, the imaging system includes a first optical path, a second optical path, a plurality of optical elements, a detector, and a processor. The first optical path guides a first beam of radiation associated with epiluminescence while the second optical path guides a second beam of radiation associated with optical coherence tomography. The plurality of optical elements transmit the first and second beams of radiation onto a sample. The detector generates optical data associated with the first and second beams of radiation returning from the sample. The optical data associated with the first and second beams of radiation correspond to substantially non-coplanar regions of the sample. The processor correlates the optical data of the first beam with the optical data of the second beam and generates an image of the sample.

### CROSS-REFERENCE TO RELATED APPLICATIONS

This application claims the benefit under 35 U.S.C. §119(e) of U.S. provisional patent application Ser. No. 61/899,673, filed Nov. 4, 2013, which is incorporated by reference herein in its entirety.

### BACKGROUND

1. Field

Embodiments of the invention relate to designs of, and methods of using, an imaging device that collects and correlates epiluminescence and optical coherence tomography data of a sample to generate enhanced surface and depth images of the sample.

2. Background

Dermatoscopes have been used for many years by medical professionals to produce images of the human epithelia for cancer detection as well as other malignant skin diseases. One of the most common uses for dermatoscopes is for the early detection and diagnosis of skin cancers, melanoma, non-melanoma skin cancers (NMSC) including Basal Cell Carcinoma (BCC) and Squamous Cell Carcinoma (SCC), and other skin diseases including Actinic Keratosis (AK) and psoriasis. The use of light on a skin's surface to enhance the visualization of the surface is known as epiluminescence microscopy (ELM).

Dermatoscopes traditionally include a magnifier (typically ×10), a non-polarized light source, a transparent plate, and a liquid medium between the instrument and the skin, thus allowing inspection of skin lesions unobstructed by skin surface reflections. Some more contemporary dermatoscopes dispense with the use of a liquid medium and instead use polarized light to cancel out skin surface reflections.

ELM alone provides surface imaging of the skin, and can even provide a three-dimensional model of the skin surface when using multiple ELM sources. However, ELM data does not provide the medical professional with any images or information beneath the surface of the skin. Such data would be useful for cancer detection and diagnosis, and locating tumors or other abnormalities below the skin surface. Lesion inspection based on ELM data alone is often unable to provide an adequate differential diagnosis and the medical professional needs to resort to excisional biopsy.

Optical Coherence Tomography (OCT) is a medical imaging technique providing depth resolved information with high axial resolution by means of a broadband light source (or a swept narrowband source) and an interferometric detection system. It has found plenty of applications, ranging from ophthalmology and cardiology to gynecology and in-vitro high-resolution studies of biological tissues. Although OCT can provide depth-resolved imaging, it typically requires bulky equipment.

### BRIEF SUMMARY

An imaging system and method for use are presented. The imaging system collects and correlates data taken using both ELM and OCT techniques from the same device.

In an embodiment, an imaging system includes a first optical path, a second optical path, a plurality of optical elements, a detector, and a processor. The first optical path guides a first beam of radiation associated with epiluminescence while the second optical path guides a second beam of radiation associated with optical coherence tomography. The plurality of optical elements transmit the first and second beams of radiation onto a sample. The detector generates optical data associated with the first and second beams of radiation that have been reflected or scattered from the sample and are received at the detector. The optical data associated with the first and second beams of radiation correspond to substantially non-coplanar regions of the sample. The processor correlates the optical data associated with the first beam of radiation with the optical data associated with the second beam of radiation and generates an image of the sample based on the correlated optical data.

In another embodiment, a handheld imaging device includes a first optical path, a second optical path, a plurality of optical elements, a detector, and a transmitter. The first optical path guides a first beam of radiation associated with epiluminescence while the second optical path guides a second beam of radiation associated with optical coherence tomography. The plurality of optical elements transmit the first and second beams of radiation onto a sample. The detector generates optical data associated with the first and second beams of radiation that have been reflected or scattered from the sample and are received at the detector. The optical data associated with the first and second beams of radiation correspond to substantially non-coplanar regions of the sample. The transmitter is designed to transmit the optical data to a computing device.

An example method is also described. In an embodiment, first optical data associated with epiluminescence imaging of a sample is received. Second optical data associated with optical coherence tomography imaging of the sample is also received, wherein the first optical data and the second optical data correspond to substantially non-coplanar regions of the sample. A processing device correlates one or more frames of the first optical data with one or more frames of the second optical data to generate correlated data. The processing device also generates an image of the sample based on the correlated data.

In an embodiment, a non-transitory computer-readable storage medium includes instructions that, when executed by a processing device, cause the processing device to perform the method disclosed above.

Further features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form a part of the specification, illustrate embodiments of the present invention and, together with the description, further serve to explain the principles of the invention and to enable a person skilled in the pertinent art to make and use the invention.

FIG. 1 illustrates an imaging system, according to an embodiment.

FIG. 2 illustrates two imaging planes with respect to a sample surface, according to an embodiment.

FIGS. 3A-D illustrate the effects of image translation and rotation.

FIGS. 4A-B illustrate the effects of out-of-plane image rotation.

FIGS. 5A-B illustrate the effects of image translation.

FIG. 6 illustrates an example method.

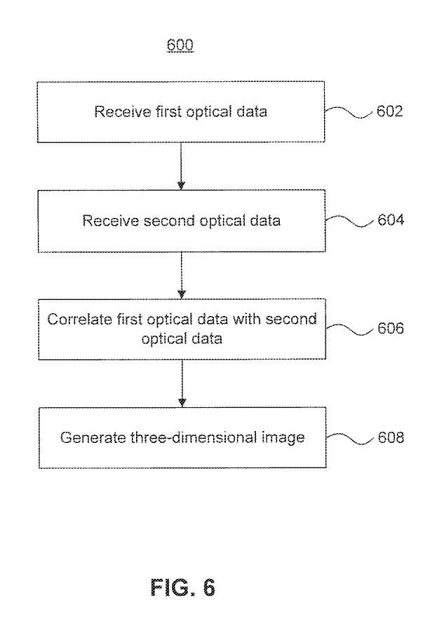

FIG. 7 illustrates another example method.

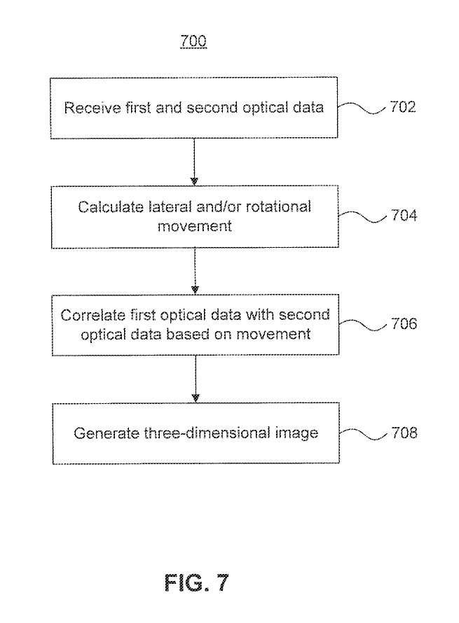

FIG. 8 illustrates an example computer system useful for implementing various embodiments.

Embodiments of the present invention will be described with reference to the accompanying drawings.

### DETAILED DESCRIPTION

Although specific configurations and arrangements are discussed, it should be understood that this is done for illustrative purposes only. A person skilled in the pertinent art will recognize that other configurations and arrangements can be used without departing from the spirit and scope of the present invention. It will be apparent to a person skilled in the pertinent art that this invention can also be employed in a variety of other applications.

It is noted that references in the specification to “one embodiment.” “an embodiment,” “an example embodiment,” etc., indicate that the embodiment described may include a particular feature, structure, or characteristic, but every embodiment may not necessarily include the particular feature, structure, or characteristic. Moreover, such phrases do not necessarily refer to the same embodiment. Further, when a particular feature, structure or characteristic is described in connection with an embodiment, it would be within the knowledge of one skilled in the art to effect such feature, structure or characteristic in connection with other embodiments whether or not explicitly described.

Embodiments herein relate to an imaging device that can be used in the study of human epithelia, and that combines data received from both ELM images and OCT images to generate enhanced three-dimensional images of a sample under study. In an embodiment, the imaging planes of the two image modalities are non-coplanar to allow for data to be captured and organized in three dimensions. The imaging device may include all of the optical elements necessary to provide two separate light paths, one for ELM light and the other for OCT light. In an embodiment, each light path may share one or more optical elements. It should be understood that the term “light” is meant to be construed broadly in this context, and can include any wavelength of the electromagnetic spectrum. In one example, the ELM light includes visible wavelengths between about 400 nm and about 700 nm, while the OCT light includes near infrared wavelengths between about 700 nm and 1500 nm. Other infrared ranges may be utilized as well for the OCT light. Additionally, either the ELM light or the OCT light may be conceptualized as a beam of radiation or a beam of radiation. A beam of radiation may be generated from one or more of any type of optical source.

The collected data from both the ELM and OCT light may be temporally and/or spatially correlated to enhance the resulting image. For example, relative movement between the imaging device and the sample might induce a linear transformation, including translation and/or rotation, on the ELM images, which may be detected and used to map the associated locations of the OCT images for accurate reconstruction of a three-dimensional model. Additionally, the relative movement between the imaging device and the sample might also lead to out-of-plane rotations on the ELM data, which may be calculated and used to take advantage of the increase in angular diversity for improving sample analysis in the OCT data. Further details regarding the correlation between the ELM and OCT images are described herein.

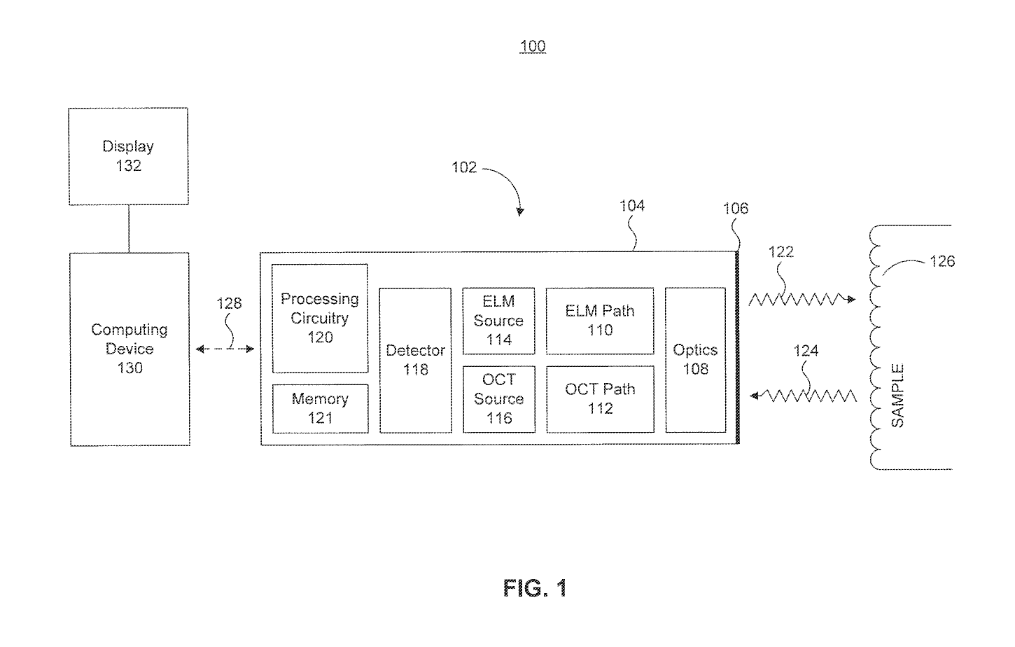

FIG. 1 illustrates an imaging system, according to an embodiment. The imaging system includes an imaging device 102, a computing device 130, and a display 132. In this example, imaging device 102 is collects data from a sample 126. Imaging device 102 and computing device 130 may be communicatively coupled via an interface 128. For example, interface 128 may be a physical cable connection, RF signals, infrared, or Bluetooth signals. Imaging device 102 may include one or more circuits and discrete elements designed to transmit and/or receive data signals across interface 128.

Imaging device 102 may be suitably sized and shaped to be comfortably held in the hand while collecting image data from sample 126. In one example, imaging device 102 is a dermatoscope. Imaging device 102 includes a housing 104 that protects and encapsulates the various optical and electrical elements within imaging device 102. In one embodiment, housing 104 includes an ergonomic design for the hand of a user. Imaging device 102 also includes an optical window 106 through which both ELM and OCT light can pass. Optical window 106 may include a material that allows a substantial portion of ELM and OCT light to pass through, in contrast to a material of housing 104 that allows substantially no ELM or OCT light to pass through. Optical window 106 may be disposed at a distal end of imaging device 102, but its location is not to be considered limiting.

In an embodiment, optical window 106 may comprise more than one portion located at different regions of imaging device 102. Each portion of optical window 106 may include a material that allows a substantial portion of a certain wavelength range of light to pass through. For example, one portion of optical window 106 may be tailored for OCT light while another portion of optical window 106 may be tailored for ELM light.

Various radiation signals are illustrated as either exiting or entering optical window 106. Transmitted radiation 122 may include both ELM light and OCT light. Similarly, received radiation 124 may include both ELM light and OCT light that has been at least one of scattered and reflected by sample 126. Other imaging modalities may be included as well, including, for example, fluorescence imaging or hyperspectral imaging.

Imaging device 102 includes a plurality of optical elements 108, according to an embodiment. Optical elements 108 may include one or more elements understood by one skilled in the art to be used with the transmission and receiving of light, such as, for example, lenses, mirrors, dichroic mirrors, gratings, and waveguides. The waveguides may include single mode or multimode optical fibers. Additionally, the waveguides may include strip or rib waveguides patterned on a substrate. The ELM light and OCT light may share the same optical elements or, in another example, different optical elements are used for each imaging modality and have properties that are tailored for the associated imaging modality.

Imaging device 102 includes an ELM path 110 for guiding the ELM light through imaging device 102 and an OCT path 112 for guiding the OCT light through imaging device 102, according to an embodiment. ELM path 110 may include any specific optical or electro-optical elements necessary for the collection and guidance of light associated with ELM light. Similarly, OCT path 112 may include any specific optical or electro-optical elements necessary for the collection and guidance of light associated with OCT light. An example of an OCT system implemented as a system-on-a-chip is disclosed in PCT application No. PCT/EP2012/059308 filed May 18, 2012, the disclosure of which is incorporated by reference herein in its entirety. In some embodiments, the OCT system implemented in at least a part of OCT path 112 is a polarization sensitive OCT (PS-OCT) system or a Doppler OCT system. PS-OCT may be useful for the investigation of skin burns while Doppler OCT may provide further data on angiogenesis in skin tumors. ELM path 110 may be coupled to an ELM source 114 provided within imaging device 102. Similarly, OCT path 112 may be coupled to an OCT source 116. Either ELM source 114 or OCT source 116 may include a laser diode, or one or more LEDs. ELM source 114 and OCT source 116 may be any type of broadband light source. In one embodiment, either or both of ELM source 114 and OCT source 116 are physically located externally from imaging device 102 and have their light transmitted to imaging device 102 via, for example, one or more optical fibers.

In one embodiment, ELM path 110 and OCT path 112 share at least a portion of the same physical path within imaging device 102. For example, a same waveguide (or waveguide bundle) is used to guide both ELM light and OCT light. Similarly, the same waveguide may be used to both transmit and receive the OCT light and ELM light through optical window 106. Other embodiments include having separate waveguides for guiding ELM light and OCT light within imaging device 102. Separate waveguides may also be used for transmitting and receiving the light through optical window 106. Each of ELM path 110 and OCT path 112 may include free space optical elements along with integrated optical elements.

ELM path 110 and OCT path 112 may include various passive or active modulating elements. For example, either optical path may include phase modulators, frequency shifters, polarizers, depolarizers, and group delay elements. Elements designed to compensate for birefringence and/or chromatic dispersion effects may be included. The light along either path may be evanescently coupled into one or more other waveguides. Electro-optic, thermo-optic, or acousto-optic elements may be included to actively modulate the light along ELM path 110 or OCT path 112.

A detector 118 is included within imaging device 102, according to an embodiment. Detector 118 may include more than one detector tailored for detecting a specific wavelength range. For example, one detector may be more sensitive to ELM light while another detector is more sensitive to OCT light. Detector 118 may include one or more of a CCD camera, photodiode, and a CMOS sensor. In an embodiment, each of detector 118, ELM path 110, and OCT path 112 are monolithically integrated onto the same semiconducting substrate. In another embodiment, the semiconducting substrate also includes both ELM source 114 and OCT source 116. In another embodiment, any one or more of detector 118, ELM path 110, and OCT path 112, ELM source 114 and OCT source 116 are included on the same semiconducting substrate. Detector 118 is designed to receive ELM light and OCT light, and generate optical data related to the received ELM light and optical data related to the received OCT light. In an embodiment, the received ELM light and OCT light has been received from sample 126 and provides image data associated with sample 126. The generated optical data may be an analog or digital electrical signal.

In an embodiment, imaging device 102 includes processing circuitry 120. Processing circuitry 120 may include one or more circuits and/or processing elements designed to receive the optical data generated from detector 118 and perform processing operations on the optical data. For example, processing circuitry 120 may correlate images associated with the ELM light with images associated with the OCT light. The correlation may be performed temporally and/or spatially between the images. Processing circuitry may also be used to generate an image of sample 126 based on the correlated data via image processing techniques. The image data may be stored on a memory 121 included within imaging device 102. Memory 121 can include any type of non-volatile memory such as FLASH memory, EPROM, or a hard disk drive.

In another embodiment, processing circuitry 120 that performs image processing techniques is included on computing device 130 remotely from imaging device 102. In this embodiment, processing circuitry 120 within imaging device 102 includes a transmitter designed to transmit data between imaging device 102 and computing device 130 across interface 128. Using computing device 130 to perform the image processing computations on the optical data to generate an image of sample 126 may be useful for reducing the processing complexity within imaging device 102. Having a separate computing device for generating the sample image may help increase the speed of generating the image as well as reduce the cost of imaging device 102.

The final generated image of sample 126 based on both the ELM data and OCT data may be shown on display 132. In one example, display 132 is a monitor communicatively coupled to computing device 130. Display 132 may be designed to project a three-dimensional image of sample 126. In a further embodiment, the three-dimensional image is holographic.

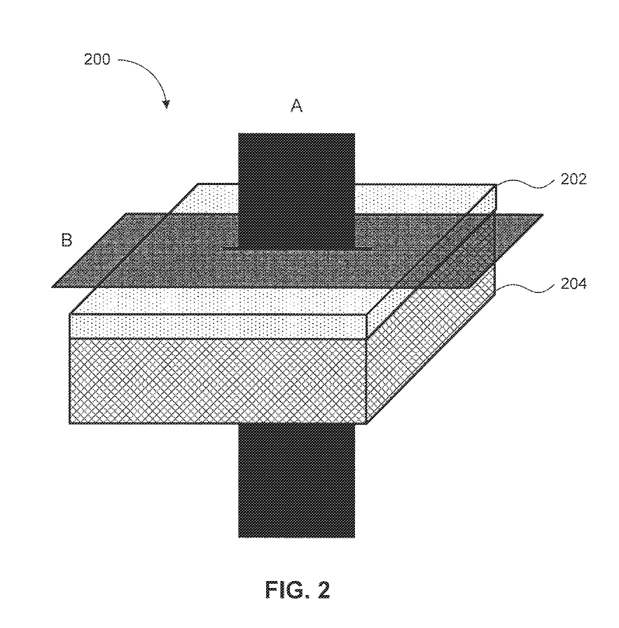

In an embodiment, imaging system 102 is capable of collecting data from two different optical signal modalities (e.g., OCT and ELM) to generate enhanced image data of a sample. The data is collected by substantially simultaneously transmitting and receiving the light associated with each signal modality. An example of this is illustrated in FIG. 2.

FIG. 2 illustrates a sample 200 being imaged by two different image modalities, according to an embodiment. Image surface B lies substantially across a surface of sample 200. In one example, image surface B corresponds to an ELM image taken of an epithelial surface 202. Image surface A corresponds to an OCT image taken through a depth of sample 200 such that the OCT image provides data on both epithelial layer 202 as well as deeper tissue 204. In one example, OCT image data along image surface A is collected by axially scanning along image surface A. These axial scans are also known as a-scans. Combining a-scans taken along image surface A provide an OCT image across image surface A within sample 200.

In an embodiment, image surface A and image surface B are non-coplanar. In one example, such as the example illustrated in FIG. 2, image surface A is substantially orthogonal to image surface B. Image surface A also intersects image surface B across a non-negligible length. Other optical sources may be used to generate more than the two image planes shown. When using and correlating two separate images, such as images taken along image surface A with images taken along image surface B, parallax effects should be accounted for. The parallax effects can be compensated along either or both of ELM path 110 and OCT path 112 within imaging device 102 using optical modulating effects. In another example, the parallax effects can be compensated for during image processing by using knowledge of exactly where the ELM light and OCT light was transmitted and collected from.

The ELM images taken on the surface of a sample can be correlated with the OCT images being taken, according to an embodiment. One advantage of this correlation is the ability to determine accurate locations of the axially-scanned portions of the OCT images based on a transformation observed in the ELM images. In an embodiment, both image modalities provide timed acquisition of all frames so that the delay between two frames is known within modalities. In another embodiment, the acquisition between at least a defined subset of frame pairs between image modalities is substantially simultaneous.

In an embodiment, imaging device 102 may be designed to allow for relative displacement between a sample under investigation and the fields of view (FOV) of both imaging modalities. However, the relative position between both FOVs should not be affected by the relative movement of imaging device 102. In one example, this behavior can be obtained through substantially rigid fixation of all optical elements used in imaging device 102. During a displacement of imaging device 102, two image sequences are produced corresponding to each image modality, whereby at least two subsets of these images can be formed by frames acquired in a substantially simultaneous way, according to an embodiment. Temporal or spatial sampling from the ELM image data must be sufficient so as to allow for non-negligible overlap between subsequent frames.

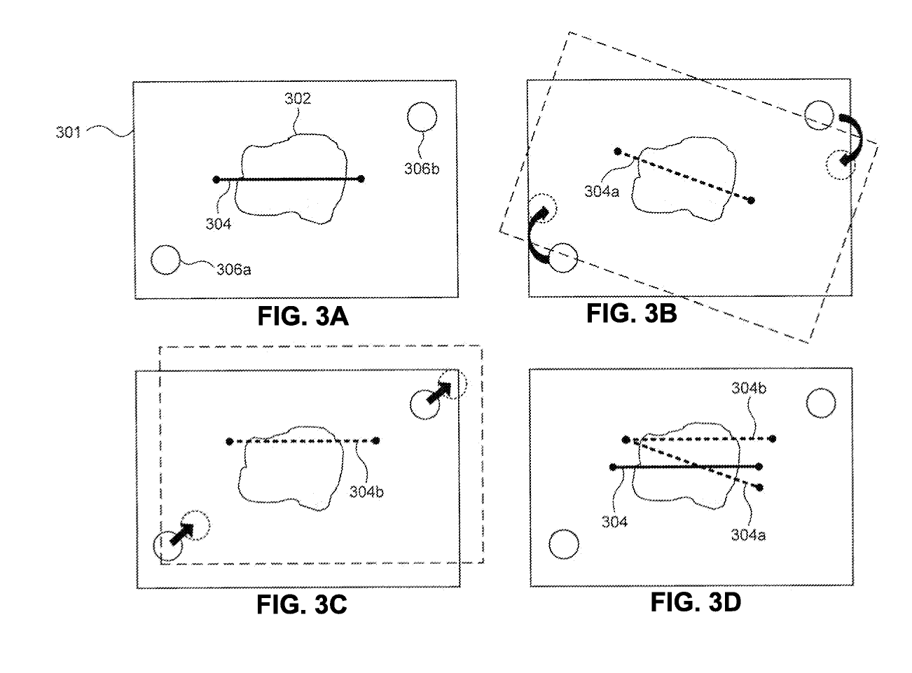

FIGS. 3A-D illustrate how both translation and rotation of ELM images can be used to track the location of the captured OCT image, according to embodiments. FIG. 3A illustrates a lesion 302 on a sample surface 301 that may be imaged, for example, using ELM data. As such, the ELM image may have a FOV that includes substantially all of sample surface 301. At substantially the same time that an ELM image is taken of sample surface 301, an OCT image 304 is taken across a portion of lesion 302. Marks 306a and 306b are used as guides to illustrate relative movement in the proceeding figures of the ELM images being taken of sample surface 301.

Estimating the location of the OCT image by using the ELM images allows for a complete and accurate data reconstruction of the skin surface and areas beneath the skin surface, according to an embodiment. Without the correlation, there is no reference for the captured OCT images, and thus reconstructing a final image is difficult.

FIG. 3B illustrates a FOV rotation being performed with regards to the ELM image captured in FIG. 3A. For example, the ELM image of sample surface 301 from FIG. 3A may be taken at a discrete time before the ELM image of sample surface 301 from FIG. 3B. In an embodiment, ELM images are continuously captured at a given frame rate to capture any changes that occur in the FOV of the ELM images. Marks 306a and 306b have shifted as a result of the rotation, and a new rotated position for OCT image 304a has also resulted. The amount of measured rotation from marks 306a and 306b should equate to the amount of rotation between original OCT image 304 and rotated OCT image 304a. In this way, image processing techniques performed on the surface data of the collected ELM image can be used to calculate the amount of rotation. For example, marks 306a and 306b may represent distinguishing features within the ELM image whose movement can be easily tracked.

FIG. 3C illustrates a translation of the FOV of the ELM image on sample surface 301. Here, marks 306a and 306b have been translated the same distance as translated OCT image 304b. FIG. 3D illustrates a mapping of potential movements for OCT image 304 based on whether a rotation occurred (rotated OCT image 304a) or a translation occurred (translated OCT image 304b), or both. In an embodiment, the movement map is continuously updated for each ELM image processed to also continuously track the movement of collected OCT images across lesion 302.

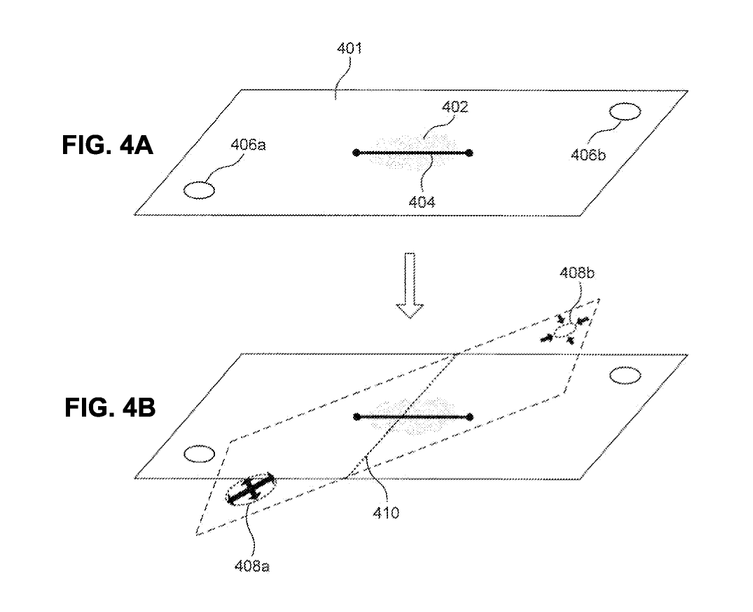

The ELM images may also be used to calculate out-of-plane rotations occurring with respect to the sample surface. FIGS. 4A-B illustrate tracking an out-of-plane rotation using ELM image data.

FIG. 4A illustrates a lesion 402 on a sample surface 401 that may be imaged, for example, using ELM data. As such, the ELM image may have a FOV that includes substantially all of sample surface 401. At substantially the same time that an ELM image is taken of sample surface 401, an OCT image 404 is taken across a portion of lesion 402. Marks 406a and 406b are used as guides to illustrate relative movement in the proceeding ELM images being taken of sample surface 401.

FIG. 4B illustrates a rotation of the FOV of the ELM image of sample surface 401 about an axis 410. The rotation results in a change in position of image feature 406a and 406b to image features 408a and 408b respectively. Specifically, mark 408a appears larger due to the rotation while mark 408b appears smaller due to the rotation (assuming a situation where the imaging device is taking the ELM images in a top-down manner with regards to FIGS. 4A-B.) The out-of-plane rotation may be calculated by using computational registration techniques, such as optical flow, between image features 406a to 408a and 406b to 408b. It should also be understood that the optical flow may be calculated using data from substantially the whole ELM image and not just within given regions of the ELM image. This calculated rotation may then be used to correct or track the position of the collected OCT image 404.

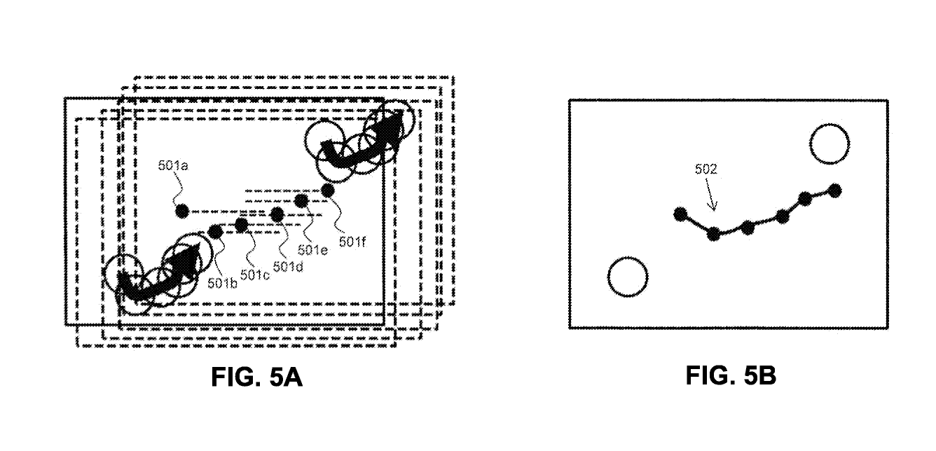

In an embodiment, the sampling rate or frame rate capture of the ELM images is higher than the capturing of the a-scans associated with the OCT images. In one example, various a-scans associated with a single OCT image are captured while a movement of imaging device 102 may cause the a-scans to no longer to be taken across a single plane. In this situation, the captured ELM images may be used to correlate the locations of the a-scans, and ultimately determine a path of the OCT image across a surface of the sample. This concept is illustrated in FIGS. 5A-5B.

FIG. 5A illustrates an example movement path of captured ELM images on a surface of a sample, and the associated movement that occurs to the captured a-scans of a single OCT image. The large squares with dashed lines represent the shifting position in time of the captured ELM images (as indicated by the large arrows) while the straight dashed lines in the center of the image represent the planes across which the OCT image is being taken through time. The large dots 501a-f on each OCT image plane represent a-scans that scan across an OCT image plane from left to right. As can be seen, the a-scans do not scan across a single plane due to the translational movement across the sample surface.

The movement of the ELM images may be used to track the positions of the a-scans of the OCT image, according to an embodiment. FIG. 5B illustrates the actual collected OCT image 502 from combining the a-scans over its crooked path. The path of OCT image 502 may be determined by correlating the positions of the a-scans 501a-e with the captured ELM images.

All of the image processing analyses described may be performed by processing circuitry 120 or remotely from imaging device 102 by computing device 130. The analysis may be done on immediately subsequent ELM images, or on pairs of images that are spaced further apart in time, independently of whether they are associated to a simultaneously acquired OCT image or not. Methods such as optical flow, phase differences between the Fourier transforms of different images, or other image registration techniques known to those skilled in the art may be applied for this purpose.

Additionally, displacements and rotations between individual pairs of registered images can be accumulated, averaged, or otherwise combined so as to produce a transformation for each acquired epiluminescence image and each OCT image relative to a reference coordinate frame, according to some embodiments. The combination of individual displacements may be performed in association with an estimation of the relative movement between imaging device 102 and plurality of optical elements 108 within imaging device 102 to help minimize errors. Such an estimation may be performed with the use of a Kalman filter or some other type of adaptive or non-adaptive filter. This may be relevant if some OCT images are not acquired substantially simultaneously to the ELM images, if sampling is non-uniform in time or if individual calculations of shift and rotation are noisy because of image quality or algorithm performance. In an embodiment, the filter used to minimize error is implemented in hardware within processing circuitry 120. However, other embodiments may have the filter implemented in software during the image processing procedure.

The various shifts and rotations computed for the ELM images may be used to merge them and to produce an ELM image having an expanded FOV. This ELM image may be stored and presented to a user on a device screen, such as display 132.

In another embodiment, the various shifts and rotations computed for the ELM images are correlated with associated OCT images and the data is merged to form a three-dimensional dataset. In an embodiment, the three-dimensional dataset offers dense sampling at a given depth beneath the surface of the sample being imaged. In another embodiment, the three-dimensional dataset offers sparse sampling at a given depth beneath the surface of the sample being imaged. In one example, data sampling occurs for depths up to 2 mm below the surface of the sample. In another example, data sampling occurs for depths up to 3 mm below the surface of the sample. The three-dimensional dataset may be rendered as a three-dimensional image of the sample and displayed on display 132. In an embodiment, the rendering is achieved using at least one of marching cubes, ray tracing, or any other 3D rendering technique known to those skilled in the art.

In an embodiment, a correlation between the ELM images and the OCT images involves the transfer of information, such as metadata, between the imaging modalities. For example, annotations and/or markers created automatically, or by a user, in one imaging modality may have their information passed on to the associated images of another imaging modality. Any spatially-related, or temporally-related, metadata from one imaging modality may be passed to another imaging modality. One specific example includes delineating tumor margins in one or more ELM images, and then passing the data associated with the delineating markers to the correlated OCT images to also designate the tumor boundaries within the OCT data. Such cross-registration of data between imaging modalities may also be useful for marking lesion limits for mohs surgery guidance or to document biopsy locations.

In an embodiment, the various captured OCT images may be used to segment the surface of the sample at the intersection between the OCT imaging plane and the sample surface. These intersection segments may be combined to develop an approximation of the topography of the sample surface. The ELM image data may then be used to “texture” the surface topology generated from the OCT data. For example, the OCT image data may be used to create a texture-less wire-mesh of a sample surface topology. The ELM image data, and preferably, though not required, the expanded FOV ELM image data may then be applied over the wire-mesh to create a highly detailed textured surface map of the sample surface. Additionally, since the OCT images provide depth-resolved data, information can also be quickly accessed and visualized regarding layers beneath the sample surface. Such information can aid healthcare professionals and dermatologists in making speedier diagnoses, and can help plan for tumor removal surgery without the need for a biopsy.

In an embodiment, local roughness parameters may be computed from the reconstructed sample surface or from the individual OCT images, and overlaid or otherwise displayed together with the reconstructed sample image. The roughness parameters may also be mapped on the reconstructed sample surface using a false-color scale or with any other visualization techniques.

In an embodiment, the collection of OCT images is triggered based on the relative movement between collected ELM images. For example, if the translation between two or more ELM images is too large, then imaging device 102 is sweeping too quickly across the sample surface and the OCT images would be blurry. In this way, OCT images are only captured during situations where the lower sampling frequency of the OCT data would not cause errors in the data collection. In another example, OCT images continue to be captured and certain images are discarded when the relative movement between captured ELM images caused too much degradation within the captured OCT image. The estimation of image motion obtained from the ELM image sequence may also be used to quantify the motion blur in both ELM images and to filter out, or at least identify, the lower quality images. In another embodiment, when there is no movement during a given period of time, a set of OCT images recorded during the time lapse can be combined for denoising and enhancement purposes, thereby improving the quality of a given OCT image. Further techniques for providing image enhancement by using the two different image modalities are contemplated as well.

In another embodiment, the three-dimensional imaging capabilities may be enhanced by including a second ELM path within imaging device 102. The second ELM path would be located separately from the first ELM path, and the difference in location between the two paths may be calibrated and leveraged to produce stereoscopic three-dimensional images of the sample surface.

In another embodiment, a three-dimensional representation of a sample surface may be generated using a single ELM path within imaging device 102. The displacement information collected between temporally sequential ELM images is used to estimate a relative point-of-view for each of the captured ELM images. A three-dimensional representation of the sample surface may be generated from combined ELM images and data regarding their associated points-of-view of the sample.

An example method 600 is described for generating a sample image based on both ELM and OCT image data of the sample, according to an embodiment. Method 600 may be performed by processing circuitry 120 within imaging device 102, or by computing device 130.

At block 602, first optical data associated with ELM is received. The first optical data may be received across a wireless interface or via hard-wired circuitry. In an embodiment, the first optical data is generated by a detector when the detector receives light associated with ELM. The ELM light received by the detector has been collected from the surface of a sample, according to one example.

At block 604, second optical data associated with OCT is received. The second optical data may be received across a wireless interface or via hard-wired circuitry. In an embodiment, the second optical data is generated by a detector when the detector receives light associated with OCT. The OCT light received by the detector has been collected from various depths of a sample, according to one example. In an embodiment, the image plane corresponding to the first optical data is non-coplanar with the image plane corresponding to the second optical data.

At block 606, one or more images of the first optical data are correlated with one or more images of the second optical data. The correlation may be performed spatially or temporally between the images from the two modalities.

At block 608, an image of the sample is generating using the correlated data from block 606. The image may be a three-dimensional representation of the sample based on combined ELM and OCT data. Surface roughness data may be calculated and overlaid with the generated image, according to an embodiment. The generated image provides data not only of the sample surface, but also at various depths beneath the sample surface, according to an embodiment.

Another method 700 is described for generating a sample image based on both ELM and OCT image data of the sample, according to an embodiment. Method 700 may be performed by processing circuitry 120 within imaging device 102, or by computing device 130.

At block 702, first and second optical data are received. The first optical data may correspond to measured ELM image data, while the second optical data may correspond to measured OCT image data. In an embodiment, the image plane corresponding to the first optical data is non-coplanar with the image plane corresponding to the second optical data.

At block 704, a translational and/or rotational movement is calculated based on temporally collected images from the first optical data. When the first optical data is ELM data, ELM images may be collected over a time period and analyzed to determine how far the images have translated or rotated. During the same time that the ELM images are collected, OCT images may also be collected. In one example, an OCT image is captured at substantially the same time as an associated ELM image.

At block 706, the first optical data is correlated with the second optical data. ELM images may be associated with OCT images that are captured at substantially the same time and that have intersecting image planes on the sample. The calculated movement of the ELM images may be used to map the movement and location of the associated OCT images. Images from the first and second optical data may be temporally or spatially correlated with one another.

At block 708, a three-dimensional image is generated based on the correlated optical data. The image may be a three-dimensional representation of the sample based on combined ELM and OCT data. For example, the various shifts and rotations computed for the ELM images can be used to map the locations of the associated OCT images, and the data is merged to form a three-dimensional model providing one or both of surface data textured with the ELM image data, and depth-resolved data from the OCT image data.

Various methods may be used to generate a model of a sample surface and depth using the combined OCT and ELM data. For example, the OCT data may be used to generate a “wire mesh” representation of the sample surface topology. The ELM data may then be applied to the wire mesh surface like a surface texture. Other examples include box modeling and/or edge modeling techniques for refining the surface topology of the sample.

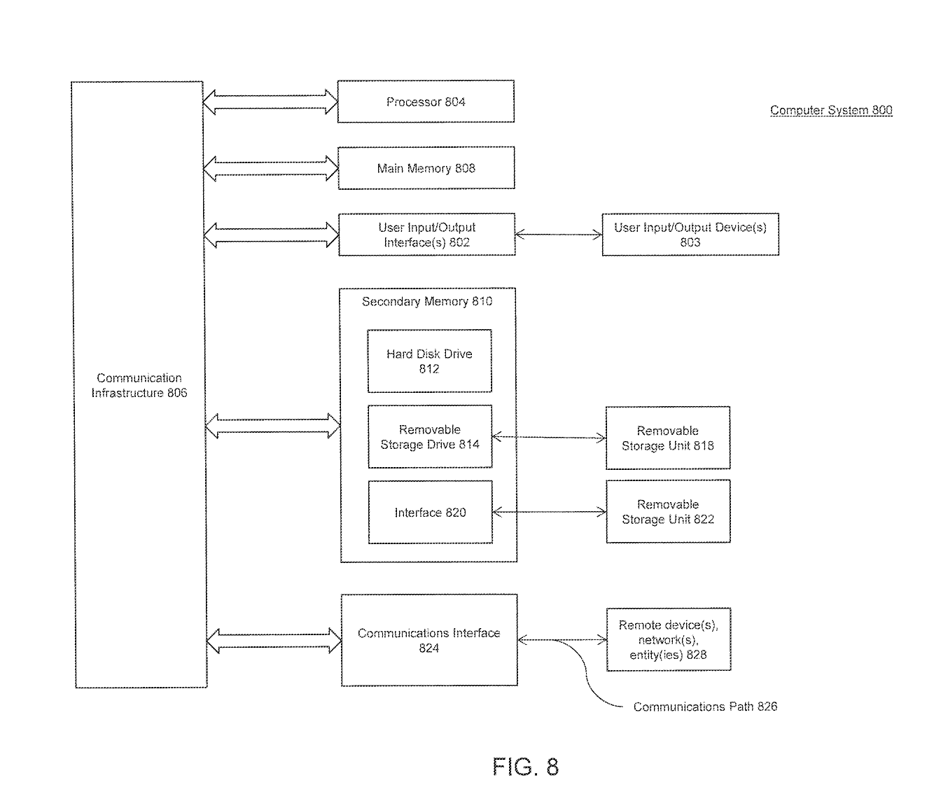

Various image processing methods and other embodiments described thus far can be implemented, for example, using one or more well-known computer systems, such as computer system 800 shown in FIG. 8.

Computer system 800 includes one or more processors (also called central processing units, or CPUs), such as a processor 804. Processor 804 is connected to a communication infrastructure or bus 806. In one embodiment, processor 804 represents a field programmable gate array (FPGA). In another example, processor 804 is a digital signal processor (DSP).

One or more processors 804 may each be a graphics processing unit (GPU). In an embodiment, a GPU is a processor that is a specialized electronic circuit designed to rapidly process mathematically intensive applications on electronic devices. The GPU may have a highly parallel structure that is efficient for parallel processing of large blocks of data, such as mathematically intensive data common to computer graphics applications, images and videos.

Computer system 800 also includes user input/output device(s) 803, such as monitors, keyboards, pointing devices, etc., which communicate with communication infrastructure 806 through user input/output interface(s) 802.

Computer system 800 also includes a main or primary memory 808, such as random access memory (RAM). Main memory 808 may include one or more levels of cache. Main memory 808 has stored therein control logic (i.e., computer software) and/or data. In an embodiment, at least main memory 808 may be implemented and/or function as described herein.

Computer system 800 may also include one or more secondary storage devices or memory 810. Secondary memory 810 may include, for example, a hard disk drive 812 and/or a removable storage device or drive 814. Removable storage drive 814 may be a floppy disk drive, a magnetic tape drive, a compact disk drive, an optical storage device, tape backup device, and/or any other storage device/drive.

Removable storage drive 814 may interact with a removable storage unit 818. Removable storage unit 818 includes a computer usable or readable storage device having stored thereon computer software (control logic) and/or data. Removable storage unit 818 may be a floppy disk, magnetic tape, compact disk, Digital Versatile Disc (DVD), optical storage disk, and any other computer data storage device. Removable storage drive 814 reads from and/or writes to removable storage unit 818 in a well-known manner.

Secondary memory 810 may include other means, instrumentalities, or approaches for allowing computer programs and/or other instructions and/or data to be accessed by computer system 800. Such means, instrumentalities or other approaches may include, for example, a removable storage unit 822 and an interface 820. Examples of the removable storage unit 822 and the interface 820 may include a program cartridge and cartridge interface (such as that found in video game devices), a removable memory chip (such as an EPROM or PROM) and associated socket, a memory stick and universal serial bus (USB) port, a memory card and associated memory card slot, and/or any other removable storage unit and associated interface.

Computer system 800 may further include a communication or network interface 824. Communication interface 824 enables computer system 800 to communicate and interact with any combination of remote devices, remote networks, remote entities, etc. (individually and collectively referenced by reference number 828). For example, communication interface 824 may allow computer system 800 to communicate with remote devices 828 over communications path 826, which may be wired and/or wireless, and which may include any combination of local area networks (LANs), wide area networks (WANs), the Internet, etc. Control logic and/or data may be transmitted to and from computer system 800 via communication path 826.

In an embodiment, a tangible apparatus or article of manufacture comprising a tangible computer useable or readable medium having control logic (software) stored thereon is also referred to herein as a computer program product or program storage device. This includes, but is not limited to, computer system 800, main memory 808, secondary memory 810, and removable storage units 818 and 822, as well as tangible articles of manufacture embodying any combination of the foregoing. Such control logic, when executed by one or more data processing devices (such as computer system 800), causes such data processing devices to operate as described herein.

Based on the teachings contained in this disclosure, it will be apparent to persons skilled in the relevant art(s) how to make and use the invention using data processing devices, computer systems and/or computer architectures other than that shown in FIG. 8. In particular, embodiments may operate with software, hardware, and/or operating system implementations other than those described herein.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

Embodiments of the present invention have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. An imaging system, comprising:
a first optical path configured to guide a first beam of radiation associated with epiluminescence microscopy;
a second optical path configured to guide a second beam of radiation associated with optical coherence tomography;
a plurality of optical elements configured to transmit the first and second beams of radiation onto a sample;
a detector configured to generate optical data associated with the first and second beams of radiation that have been reflected or scattered from the sample and are received at the detector, wherein the optical data associated with the first and second beams of radiation correspond to substantially non-coplanar regions of the sample; and
a processor configured to:
correlate the optical data associated with the first beam of radiation with the optical data associated with the second beam of radiation, and
generate an image of the sample based on the correlated optical data.

2. The imaging system of claim 1, wherein the first and second optical paths comprise one or more optical fibers.

3. The imaging system of claim 1, wherein the first and second optical paths comprise one or more waveguides patterned on a substrate.

4. The imaging system of claim 1, wherein the first optical path, the second optical path, the plurality of optical elements, and the detector are disposed within a handheld imaging device.

5. The imaging system of claim 4, wherein a portion of the handheld imaging device is substantially transparent to the first and second beam of radiation, and the plurality of optical elements are configured to transmit the first and second beams of radiation through the portion of the handheld imaging device.

6. The imaging system of claim 4, wherein a first portion of the handheld imaging device is substantially transparent to the first beam of radiation, and a second portion of the handheld imaging device is substantially transparent to the second beam of radiation, and wherein a first portion of the plurality of optical elements are configured to transmit the first beam of radiation through the first portion and a second portion of the plurality of optical elements are configured to transmit the second beam of radiation through the second portion.

7. The imaging system of claim 4, wherein the processor is included within the handheld imaging device.

8. The imaging system of claim 4, wherein the processor is included within a computing device that is communicatively coupled to the handheld imaging device.

9. The imaging system of claim 1, wherein the substantially non-coplanar regions of the sample are substantially orthogonal regions of the sample.

10. The imaging system of claim 1, wherein the first optical path and the second optical path share at least a portion of the same physical path.

11. The imaging system of claim 1, wherein the detector comprises at least one of a CCD camera, photodiode, and CMOS sensor.

12. The imaging system of claim 1, wherein the processor is further configured to:
analyze temporally sequential optical data associated with the first beam of radiation and use the temporally sequential optical data of the first beam of radiation to calculate at least one of a translational movement, and a rotation of the device with respect to the surface of the sample.

13. The imaging system of claim 12, wherein the processor is configured to not use the optical data associated with the second beam of radiation for generating the image when the translational movement across the surface of the sample exceeds a threshold value.

14. The imaging system of claim 12, wherein the processor is further configured to correlate locations of one or more images associated with the first beam of radiation with locations of one or more images associated with the second beam of radiation based on at least one of the calculated lateral movement and rotation.

15. The imaging system of claim 14, wherein the image generated by the processor is a three-dimensional image of the sample that provides data on the surface of the sample as well as data throughout a depth beneath the sample's surface.

16. The imaging system of claim 15, wherein the data on the surface of the sample comprises data associated with a roughness of the surface of the sample.

17. The imaging system of claim 1, wherein data associated with the one or more images associated with the first beam of radiation is passed to the one or more images associated with the second beam of radiation, or vice versa.

18. The imaging system of claim 17, wherein the data includes an annotation, a marker, or metadata.

19. The imaging system of claim 1, wherein the second optical path is configured to guide the second beam of radiation associated with polarization sensitive optical coherence tomography.

20. The imaging system of claim 1, wherein the second optical path is configured to guide the second beam of radiation associated with Doppler optical coherence tomography.

21. A method comprising:
receiving first optical data associated with epiluminescence microscopy imaging of a sample;
receiving second optical data associated with optical coherence tomography imaging of the sample, wherein an orientation of an image plane associated with the first optical data with respect to an image plane associated with the second optical data on a surface of the sample is non-coplanar,
correlating, using a processing device, one or more images of the first optical data with one or more images of the second optical data to generate correlated data; and
generating, using the processing device, an image of the sample based on the correlated data.

22. The method of claim 21, further comprising:
generating the first optical data using a detector configured to receive a first beam of radiation associated with epiluminescence from the sample; and
generating the second optical data using a detector configured to receive a second beam of radiation associated with optical coherence tomography from the sample.

23. The method of claim 21, wherein the correlating comprises temporally correlating one or more frames of the first optical data with one or more frames of the second optical data to generate temporally correlated data.

24. The method of claim 21, further comprising:
analyzing temporally sequential first optical data and using the temporally sequential first optical data to calculate at least one of a translational movement and a rotation with respect to the surface of the sample.

25. The method of claim 24, further comprising expanding a field of view of the generated image across the surface of the sample based on the calculated lateral movement.

26. The method of claim 24, wherein the correlating comprises correlating locations of one or more image frames associated with the first optical data with locations of one or more image frames associated with the second optical data based on the calculated translational and rotational movement between the imaging device and the sample.

27. The method of claim 21, wherein the correlating comprises passing data associated with the one or more images of the first optical data to the one or more images of the second optical data, or vice versa.

28. The method of claim 27, wherein the data includes an annotation, a marker, or metadata.

29. The method of claim 21, wherein the generating comprises generating a three-dimensional image of the sample.

30. The method of claim 29, further comprising analyzing a roughness of the surface of the sample using the generated three-dimensional image.

31. The method of claim 29, further comprising analyzing tumor malignancy data associated with a depth beneath the surface of the sample.

32. A handheld imaging device, comprising:
a first optical path configured to guide a first beam of radiation associated with epiluminescence microscopy;
a second optical path configured to guide a second beam of radiation associated with optical coherence tomography;
a plurality of optical elements configured to transmit the first and second beams of radiation onto a sample;
a detector configured to generate optical data associated with the first and second beams of radiation that have been reflected or scattered from the sample and are received at the detector, wherein the optical data associated with the first and second beams of radiation correspond to substantially non-coplanar regions of the sample; and
a transmitter configured to transmit the optical data to a computing device.

33. A non-transitory computer-readable storage medium having instructions stored thereon that, when executed by a processing device, cause the processing device to perform a method comprising:
receiving first optical data associated with epiluminescence microscopy imaging of a sample;
receiving second optical data associated with optical coherence tomography imaging of the sample, wherein the first optical data with respect to the second optical data correspond to substantially non-coplanar regions of the sample;
correlating one or more frames of the first optical data with one or more frames of the second optical data to generate correlated data; and
generating an image of the sample based on the correlated data.
